(19) United States
(12) Patent Application Publication
(10) Pub. No.: US 2026/0350787 A1
(43) Pub. Date: Nov. 5, 2026

(54) DYNAMIC IMAGE ANALYSIS APPARATUS, METHOD, AND NON-TRANSITORY COMPUTER-READABLE RECORDING MEDIUM STORING PROGRAM

(71) Applicant: Konica Minolta, Inc., Tokyo (JP)

(72) Inventor: Kojiro Ono, Chiba (JP)

(73) Assignee: Konica Minolta, Inc., Tokyo (JP)

(21) Appl. No.: 19/194,347

(22) Filed: Apr. 30, 2025

(30) Foreign Application Priority Data
May 1, 2024 (JP) 2024-074399

(51) Int. Cl. G06T 7/00; A61B 6/00

(52) U.S. Cl. CPC G06T 7/0012; A61B 6/5217; G06T 2207/10116; G06T 2207/20016; G06T 2207/20021; G06T 2207/20056; G06T 2207/30061

(57) ABSTRACT

A dynamic image analysis apparatus according to an embodiment of the present disclosure includes: a hardware processor that performs an acquisition of a dynamic image of an imaged person who has been imaged by dynamic imaging; and a hardware processor that performs a non-stationary spectrum analysis on the dynamic image having been acquired by the hardware processor that performs the acquisition.

### CROSS REFERENCE TO RELATED APPLICATIONS

The entire disclosure of Japanese Patent Application No. 2024-074399, filed on May 1, 2024, is incorporated herein by reference in its entirety.

### BACKGROUND

### Technological Field

The present disclosure relates to a dynamic image analysis apparatus, a method, and a non-transitory computer-readable recording medium storing a program.

### Description of Related Art

Dynamic imaging is performed in which a radiation generating apparatus repeatedly emits radiation pulses at a period (pulse period) of a plurality of times per unit time (for example, 15 times per second) for a predetermined time (duration) while an emission instruction is given, and in which a radiation detection apparatus reads out, as a signal value (intensity), the amount of electric charge generated according to the dose of radiation received through the subject. By the dynamic imaging, a dynamic image including a plurality of (a series of) still images whose imaging times are different from each other for each pulse period is captured. The period at which still images are captured is called the frame rate, and is equal to the period of radiation pulses. It is a common practice that doctors diagnose diseases based on captured dynamic images. A doctor can diagnose a lung disease or a heart disease based on the movement of the lungs or the heart by dynamic imaging of organs such as the lungs and the heart. In addition, a doctor can perform a diagnosis based on the movement of a joint by dynamic imaging of a bone.

An analysis is performed in which a fast Fourier transform (FFT) is performed on a dynamic image and a specific spectrum such as a periodic signal synchronized with a heartbeat is extracted. A specific frequency spectrum can be extracted by the FFT. When a frequency synchronized with the cardiac motion is extracted as a frequency spectrum to be extracted from a dynamic image by the FFT, the contraction and dilation of a blood vessel associated with the cardiac motion in the dynamic image can be extracted. Information useful for the diagnosis of pulmonary embolism can be obtained by detecting a region where the contraction and dilation of a blood vessel associated with the cardiac motion decrease. The region where a signal decreases can be detected by, for example, a difference in the signal change amount from a reference frame (for example, Japanese Patent Publication Laid-Open No. 2023-121104 and Japanese Patent Publication Laid-Open No. 2022-095871).

In a dynamic image, the contraction and dilation of a blood vessel (cardiac induced vessel dilation) occur in association with the cardiac motion and the thickness of the blood vessel and the amount of blood change due to the contraction and dilation of the blood vessel, whereby the changes are detected as changes in the X-ray intensity to be detected by an X-ray detection apparatus. Since the pulmonary artery involves significant changes in the contraction and dilation associated with the cardiac motion, a change in the contraction and dilation of a blood vessel can be detected by performing the FFT on a change in the X-ray intensity acquired from a dynamic image to extract a frequency synchronized with the cardiac motion.

On the other hand, in the lung field (anatomical lung parenchyma) on an image which is dominated by capillaries (peripheral blood vessels), the contraction and dilation of a blood vessel synchronized with the cardiac motion occur, but it is considered that a change in the X-ray intensity is attenuated with respect to a change in the intensity of the pulmonary artery.

In addition, when the tissue property of the lung parenchyma is in a state of being not uniform due to a lung disease or the like, at least one of resonance, attenuation, and reflection occurs in vibration propagation due to the contraction and dilation of a blood vessel. The phenomena of resonance, attenuation, and reflection in vibration propagation are considered to be observed as non-stationary signals.

As described above, since it is considered that biological signals (the contraction and dilation of a blood vessel associated with the cardiac motion) include a non-stationary signal, the present inventors have newly noticed that even when a stationary spectrum analysis such as the FFT is performed on a dynamic image, information related to a non-stationary signal cannot be extracted. Accordingly, it is considered that, for example, a blood flow signal related to capillaries (peripheral blood vessels) which is observed as a non-stationary signal cannot be extracted by a conventional method, and that useful information for peripheral pulmonary embolism cannot be obtained.

### SUMMARY

A dynamic image analysis apparatus according to an embodiment of the present disclosure includes: a hardware processor that performs an acquisition of a dynamic image of an imaged person who has been imaged by dynamic imaging; and a hardware processor that performs a non-stationary spectrum analysis on the dynamic image having been acquired by the hardware processor that performs the acquisition.

A dynamic image analysis method according to an embodiment of the present disclosure includes: acquiring a dynamic image of an imaged person who has been imaged by dynamic imaging; and performing a non-stationary spectrum analysis on the acquired dynamic image.

In a non-transitory computer-readable recording medium storing a dynamic image analysis program according to an embodiment of the present disclosure, the dynamic image analysis program causes a computer to execute: acquiring a dynamic image of an imaged person who has been imaged by dynamic imaging; and performing a non-stationary spectrum analysis on the acquired dynamic image.

Note that, these generic or specific aspects may be implemented as a system, an apparatus, a method, an integrated circuit, a computer program, or a recording medium, or any selective combination of a system, an apparatus, a method, an integrated circuit, a computer program, and a recording medium.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating the configuration of a dynamic image analysis apparatus;

FIG. 2 is a functional block diagram of a processing circuit;

FIG. 3 illustrates exemplary positions of ROIs;

FIG. 4 illustrates exemplary biological signals for the ROIs, respectively;

FIG. 5 illustrates an exemplary scalogram;

FIG. 6 illustrates another exemplary scalogram;

FIG. 7 illustrates an exemplary frequency spectrum;

FIG. 8 illustrates another exemplary frequency spectrum;

FIG. 9 is a flowchart of a program that is executed by the processing circuit;

FIG. 10 illustrates an image of division into blocks;

FIG. 11 is a flowchart of a program for diagnosing emphysema;

FIG. 12 illustrates an example in which a high-frequency band is selected from a calculated frequency spectrum for blocks;

FIG. 13 illustrates an example in which the calculated intensity of the high-frequency band is illustrated for each block;

FIG. 14 illustrates an example in which a low-frequency band is selected from a calculated frequency spectrum for blocks; and

FIG. 15 illustrates an example in which the calculated intensity of the low-frequency band is illustrated for each block.

### DETAILED DESCRIPTION OF EMBODIMENTS

Hereinafter, one or more embodiments of the present invention will be described with reference to the drawings. However, the scope of the invention is not limited to the disclosed embodiments.

Hereinafter, an embodiment of the present disclosure will be described in detail with reference to the accompanying drawings as appropriate.

### <Dynamic Image Analysis Apparatus>

The configuration of a dynamic image analysis apparatus 100 according to an embodiment of the present disclosure will be described.

### [Configuration]

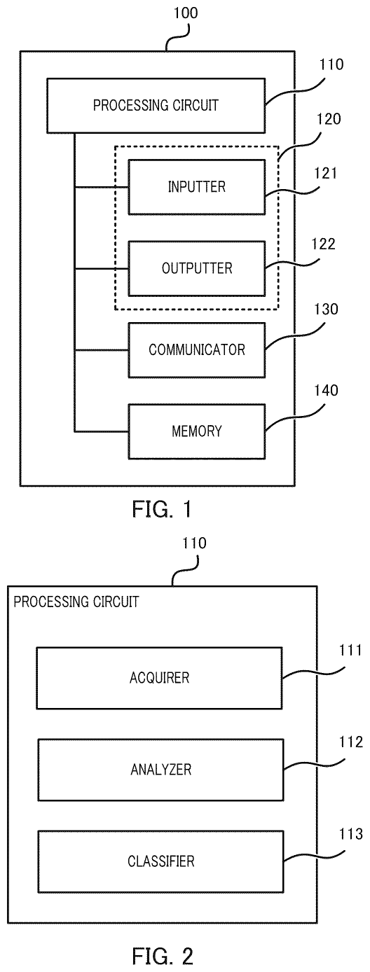

FIG. 1 is a diagram illustrating the configuration of the dynamic image analysis apparatus 100.

The dynamic image analysis apparatus 100 includes a processing circuit 110, an inputter/outputter 120, a communicator 130, and a memory 140. The inputter/outputter 120 includes an inputter 121 and an outputter 122. The inputter 121 and the outputter 122 may be integrated. In a case where an input and an output are performed via the communicator 130, the inputter/outputter 120 may be omitted.

The processing circuit 110 is constituted by a central processing unit (CPU), a graphics processing unit (GPU), an application specific integrated circuit (ASIC), a field programmable gate array (FPGA), and the like, and may include a neural network. The processing circuit 110 performs, based on an inputted medical image, a non-stationary spectrum analysis on the medical image. Details of the processing circuit 110 will be described later.

The inputter 121 includes at least one of a touch screen, a keyboard, a mouse, a microphone, and the like, and receives an input based on an operation by a user (a doctor, a radiology technician, or the like).

The outputter 122 includes at least one of a display, a speaker, a printer, and the like, and outputs a result of a non-stationary spectrum analysis performed by the processing circuit 110 to the outside.

The communicator 130 communicates with an external apparatus via a bus, a local area network (LAN), the Internet, a virtual private network (VPN), a public line, or the like wirelessly or in a wired manner. The communicator 130 communicates with a hospital information system (HIS), a radiology information system (RIS), a picture archiving and communication system (PACS), a dynamic imaging apparatus, and the like.

The memory 140 is constituted by a read only memory (ROM), a random access memory (RAM), an erasable programmable ROM (EPROM), an electrically EPROM (EEPROM), a hard disk drive (HDD), and the like, and stores dynamic images, various programs, and the like.

FIG. 2 is a functional block diagram of the processing circuit 110.

The processing circuit 110 includes an acquirer 111, an analyzer 112, and a classifier 113.

The acquirer 111 acquires a dynamic image. A dynamic image may be acquired from an external system such as an RIS via the communicator 130 based on an input from the outside via the inputter 121 or the communicator 130, or may be acquired from the memory 140. A dynamic image is, for example, a dynamic image obtained by imaging the lungs.

The analyzer 112 perform a non-stationary spectrum analysis based on a dynamic image having been acquired by the acquirer 111. Details thereof will be described later.

The classifier 113 classifies dynamic images based on analysis results of the analyzer 112. Details thereof will be described later.

### [Dynamic Image]

A dynamic image is constituted by a plurality of (a series of) still images whose imaging times are different from each other for each pulse period. A dynamic image is captured by dynamic imaging in which a dynamic imaging apparatus repeatedly emits radiation pulses at a period (pulse period) of a plurality of times per unit time (for example, 15 times per second) for a predetermined time (duration) while an emission instruction is given, and in which a radiation detection apparatus reads out, as a signal value (intensity), the amount of electric charge generated according to the dose of radiation received through the subject. An intensity I of X-rays after passing through a material can be obtained by the following equation

(1)

I=I0/exp(−μx) (equation 1),
where I0 is the intensity of X-rays incident on the material, μ is the linear attenuation coefficient, ρ is the material density, and x is the depth of the material. Here, the linear attenuation coefficient μ [1/cm] is expressed by

(2)

μ=μmρ (equation 2),
where μm is the mass attenuation coefficient [cm²/g] and p is the material density [g/cm³], and thus,

(3)

I=I0/exp(−μmρx) (equation 3)

Dynamic imaging is performed while a subject holds his/her breath, and thus, it is possible to obtain a dynamic image in which the subject and the thickness (volume) of the lungs are constant. The mass attenuation coefficient μm is constant and the X-ray emission intensity I0 of each pulse for performing dynamic imaging is also constant. As a result, according to the equation (3), when the thickness x of the lungs is constant, the intensity I of imaged X-rays is related to the density ρ of the lungs. It is presumed that the density of the lungs is influenced by a change in a biological signal, for example, a state in which a blood vessel of the lungs dilates or contracts in association with the cardiac motion. That is, it is presumed that a temporal change in the X-ray intensity in a dynamic image is influenced by a change in a biological signal, for example, the contraction and dilation of a blood vessel associated with the cardiac motion.

That is, a dynamic image that is acquired by the acquirer 111 is a dynamic image obtained by dynamic imaging while a subject holds his/her breath.

### [Biological Signal]

A biological signal is acquired from a dynamic image. A biological signal to be acquired is the X-ray intensity for each pixel (element constituting a detector of an imaging apparatus) of the detector in each frame. A biological signal may be acquired for a region of interest (ROI) in a dynamic image, for example, for each region such as a pulmonary artery portion, a peripheral blood vessel portion, an upper lobe, a middle lobe, and a lower lobe. A biological signal represents a temporal change in the X-ray intensity (time-intensity characteristics).

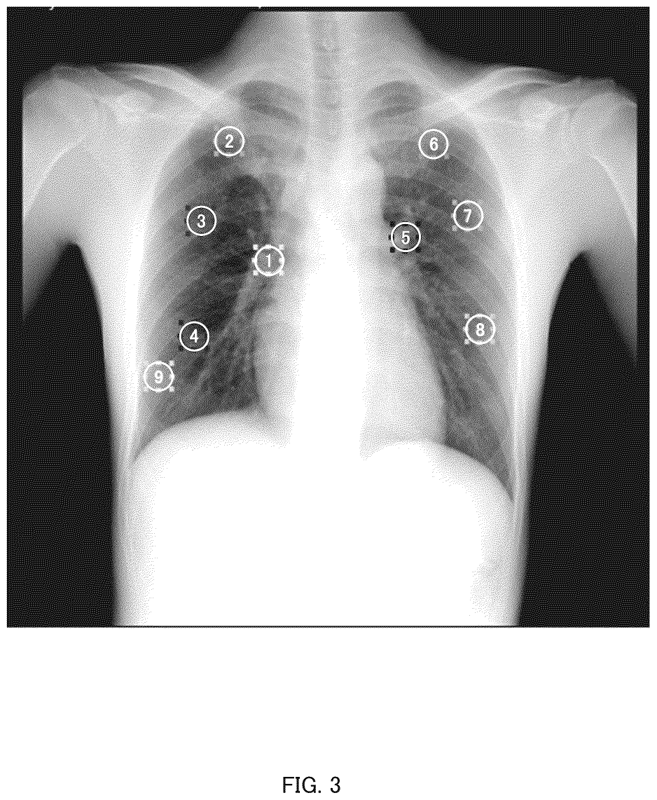

FIG. 3 is a diagram illustrating exemplary positions of ROIs. The ROIs are, for example, ID1 for right pulmonary central artery, ID2 for right pulmonary upper lobe (upper lung field, S1), ID3 for right pulmonary upper lobe (middle lung field, S3), ID4 for right middle lode (lower lung field, S4), ID5 for left pulmonary central artery, ID6 for left pulmonary upper lobe (upper lung field, S1+2), ID7 for left pulmonary upper lobe (middle lung field, S3), ID8 for left pulmonary lower lobe (lower lung field, S8), and ID9 for right pulmonary lower lobe (lower lung field, S8). The positions of ROIs and the number of ROIs are arbitrary and are not limited to nine ROIs illustrated in FIG. 3.

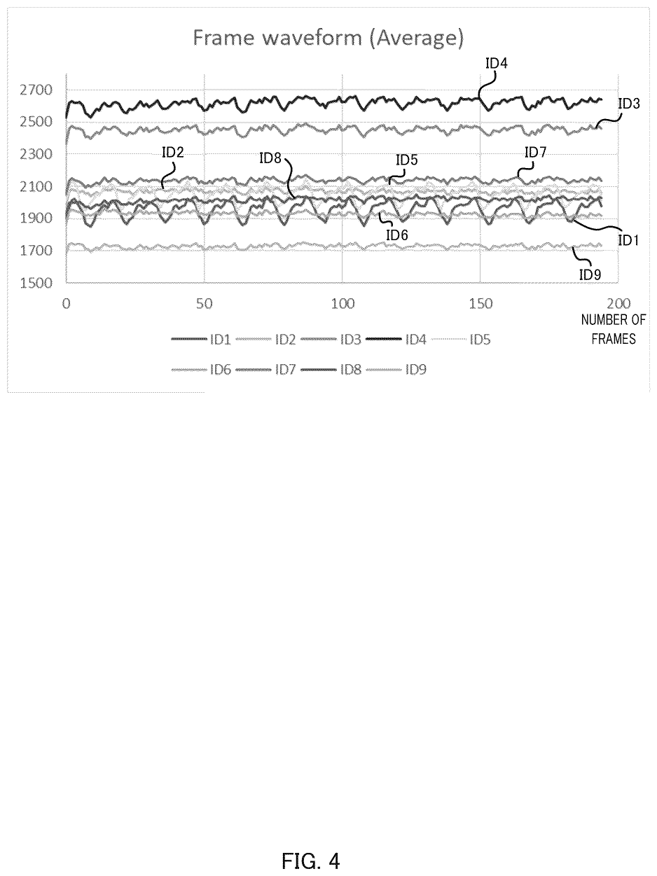

FIG. 4 illustrates exemplary biological signals for the ROIs, respectively.

The analyzer 112 performs an analysis on an acquired biological signal. Specifically, the analyzer 112 logarithmically transforms the X-ray intensity of an acquired dynamic image, divides the logarithmically transformed X-ray intensity into blocks, and performs an analysis on the dynamic image, which has been divided into the blocks, for each block. The division into blocks is processing of, for example, averaging the X-ray intensity for each of a plurality of pixels of a detector. For example, the pixel of the detector is 0.4 mm square, and the block size of the blocks is 3 mm square. The analyzer 112 may configure ROIs as blocks and performs an analysis on only the ROIs.

As the analysis, a stationary spectrum analysis or a non-stationary spectrum analysis is used. A ROI may be a predetermined region. Blocks in a dynamic image may be configured as ROIs, respectively, or a plurality of blocks in a dynamic image may be configured as one ROI. A ROI may be an organ (for example, the entire lungs, an upper lobe portion, a middle lobe portion, a lower lobe portion, and the entire heart).

A signal can be mathematically expressed as the sum (composite) of Ai sin ωit, that is,

$${\sum }_{i = 1}^{n} {A}_{i} sin {ω}_{i} t , \qquad [ 1 ]$$

where Ai is the amplitude of each signal and ωi is the angular rate of each signal. A stationary signal is a signal in which every Ai and every ωi do not change regardless of time, and a non-stationary signal is a signal in which at least one Ai or ωi changes with time. That is, when a signal in which at least one of the frequency and the amplitude changes with time is included, the signal is a non-stationary signal. In addition, regarding all of the included signals, a signal in which both the frequency and the amplitude do not change regardless of time is a stationary signal.

The stationary spectrum analysis is a technique of expressing a signal as the sum of frequencies having a constant amplitude, and is signal processing useful for a case where a stationary signal or a biological signal is assumed to be a stationary signal. As the stationary spectrum analysis, the FFT is widely used. When a specific frequency component of a stationary signal is extracted from a dynamic image by the FFT, a biological signal of the extracted frequency component can be grasped. For example, when a frequency component corresponding to the cardiac motion is extracted, the contraction and dilation of a blood vessel associated with the cardiac motion in a dynamic image can be grasped.

Since Fourier transforms including the FFT do not provide time information, the non-stationary spectrum analysis in which an analysis including time information is performed is suitable as an analysis on a non-stationary signal in which the frequency or the amplitude changes with time.

The contraction and dilation of a blood vessel are induced by the cardiac motion and are therefore a signal that generally changes periodically, but it cannot be said that the same fluctuation is repeated every time in the cardiac motion due to, for example, arrhythmia or the like. Accordingly, it is satisfactory to assume that the contraction and dilation of a blood vessel is a non-stationary signal.

In addition, the pulmonary vessels branch off from the pulmonary artery, and capillaries are perfused with blood, gas exchange by the alveoli occurs, and blood circulates to the pulmonary veins. The contraction and dilation of capillaries are a signal in which a signal induced by the cardiac motion is attenuated. Further, since the blood flow in the pulmonary veins is a steady flow and has a lower flow velocity than that in the pulmonary artery, the pulmonary veins do not contract and dilate. That is, the contraction and dilation of a pulmonary vessel (cardiac induced vessel dilation) is a shear wave whose hypocenter is the pulmonary artery on a dynamic image, and the vibration propagation thereof should be assumed to be a non-stationary signal because an attenuation signal is included on the lung parenchyma including different tissues such as the pulmonary artery and capillaries.

That is, since it is satisfactory to assume the cardiac motion itself as a non-stationary signal and the vibration propagation is also a non-stationary signal, it can be said that a biological signal extracted from a dynamic image is a non-stationary signal.

In addition, in a dynamic image, a change in the density of the lungs with respect to the irradiation direction is detected, and it is understood that the tissue of the lungs is not uniform because the pulmonary artery, capillaries, and pulmonary veins are included in the irradiation direction. Then, in a state in which the tissue of the lungs is not uniform, resonance, attenuation, and/or reflection occur(s) in the blood flow of the lungs, but the degree(s) of the resonance, attenuation, and/or reflection in the blood flow change(s) with time. That is, with respect to the blood flow in a certain ROI, it is satisfactory to assume the cardiac motion itself as a non-stationary signal, and in addition, resonance, attenuation, and/or reflection change(s) with time. Accordingly, it is appropriate to assume that a biological signal in each ROI is also a non-stationary signal.

Accordingly, the stationary spectrum analysis on biological information is a simple analysis in which an analysis is performed on a stationary signal portion included in a non-stationary signal, and it is useful that the analyzer 112 performs the non-stationary spectrum analysis on the biological information in order to analyze details of the biological information.

The non-stationary spectrum analysis is signal processing useful for a non-stationary signal, and is a time-frequency analysis, a multi-resolution analysis, or an analysis technique in which the time-frequency analysis and the multi-resolution analysis are combined. Examples of the non-stationary spectrum analysis include a wavelet transform, a windowed FFT, and a Wigner distribution.

When resonance occurs in a stationary signal, a stationary signal is attenuated, or reflection occurs in a stationary signal, the stationary signal becomes a non-stationary signal. Since resonance, attenuation, and/or reflection occur(s) in the blood flow of the lungs in a state in which the tissue of the lungs is not uniform due to a lung disease or the like, it is possible to obtain information useful for a lung disease by performing a non-stationary spectrum analysis on a dynamic image.

Generally, a low-frequency signal involves a gentle temporal change, and a high-frequency signal involves a sharp temporal change. In an analysis, the time resolution and the frequency resolution have a trade-off relationship (uncertainty principle), but the wavelet transform is suitable for the analysis on the signals described above because it is possible to perform a transform in which the frequency resolution is prioritized over the time resolution in a low-frequency region and the time resolution is prioritized over the frequency resolution in a high-frequency region. In the following description, an example in which the wavelet transform is performed as the non-stationary spectrum analysis will be described.

### [Wavelet Transform]

The wavelet transform is a technique of expressing a mother wavelet function as a sum obtained by compressing/expanding (scaling) and time-shifting (shifting) the mother wavelet function and performing an addition. The result of the wavelet transform is displayed as time-frequency characteristics, that is, as a scalogram in which the horizontal axis represents time, the vertical axis represents frequency, and the intensity (wavelet coefficient) of each frequency component is displayed in color.

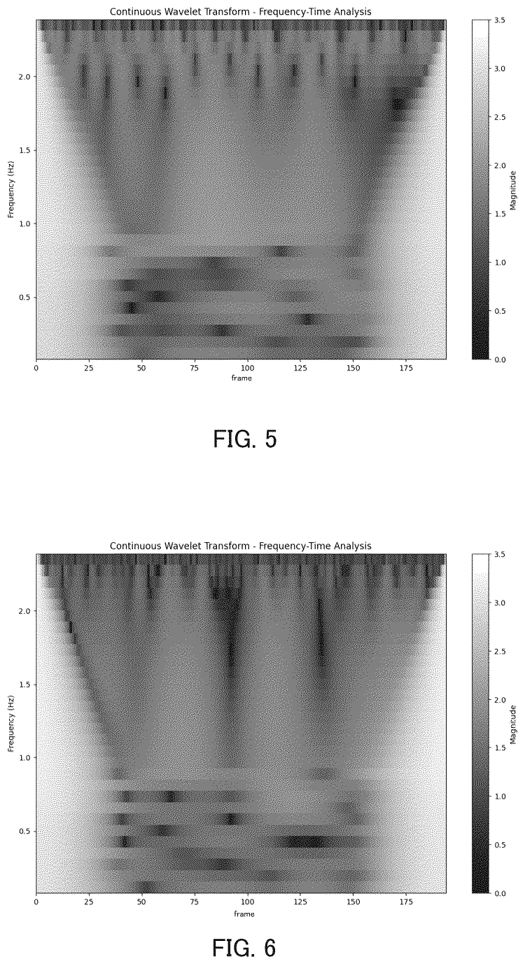

FIG. 5 illustrates an exemplary scalogram (time-frequency characteristics) in the ID1 in FIG. 3. Further, FIG. 6 illustrates an exemplary scalogram in the ID3 in FIG. 3. FIGS. 5 and 6 are displayed with concentrations corresponding to the intensities at the time and the frequency, but may be outputted with colors corresponding to the intensities at the time and the frequency.

The frequency-intensity characteristics (frequency spectrum) can be generated (calculated) by reducing the dimension of the scalogram. The maximum value, the average value, or the sum of wavelet coefficients (intensities) for frequency components is calculated for a time from a first time to a second time of the scalogram to generate a frequency spectrum. For example, in a case where a dynamic image includes 120 frames in total, the maximum value, the average value, or the sum of the intensities may be calculated based on the 21st to the 100th frames. A frequency spectrum can also be generated for a plurality of times. The generated frequency spectrum is displayed, for example, with the horizontal axis representing frequency and the vertical axis representing intensity. The intensity indicates at least one of the maximum value, the average value, and the sum. A plurality of intensities, for example, the maximum value and the average value may also be displayed.

In a scalogram dimension reduction technique, a feature amount may be calculated from a covariance matrix by using a principal component analysis (PCA).

Intensity information may be the intensity of each frequency or a ratio (frequency distribution of energy) of the intensity of each frequency to the intensities of all frequencies.

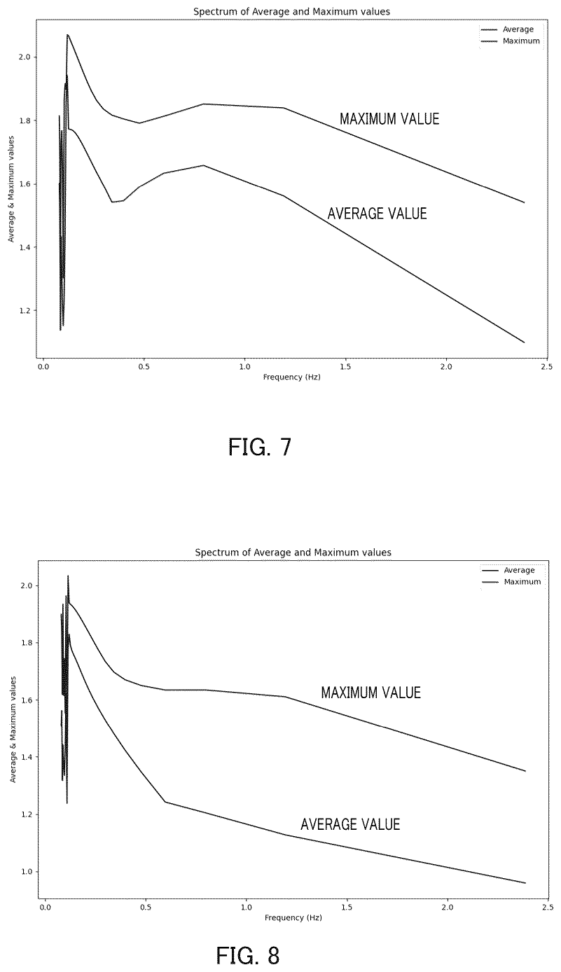

FIG. 7 illustrates an exemplary frequency spectrum in the ID1 in FIG. 3, that is, a frequency spectrum generated from the scalogram in FIG. 5. FIG. 8 illustrates an exemplary frequency spectrum in the ID3 in FIG. 3, that is, a frequency spectrum generated from the scalogram in FIG. 6. The solid lines indicate the average and the dotted lines indicate the maximum values. In FIGS. 7 and 8, the intensities of one ROI are indicated, but the intensities of a plurality of ROIs may also be simultaneously displayed. In a case where regions A to C are set as ROIs, for example, the region A may be displayed in red, the region B may be displayed in yellow, and the region C may be displayed in blue, and the maximum value in each region may be displayed with a dotted line and the average in each region may be displayed with a solid line.

### [Classification]

The classifier 113 can provide information useful for the diagnosis of a disease by performing classification based on a result of a non-stationary spectrum analysis on a dynamic image divided into blocks. The classifier 113 can provide information useful for the diagnosis of a lung disease or a heart disease by, for example, performing classification based on at least one of a scalogram, which is a result of the wavelet transform, and a frequency spectrum. The classifier 113 can perform classification based on the intensity of an arbitrary frequency band of a frequency spectrum. The frequency band for which the intensity is calculated is arbitrary and selectable.

The information useful for the diagnosis is, for example, qualitative diagnoses on a lung disease and a heart disease. The information useful for the diagnosis includes information as to whether a disease is malignant or benign, information as to what kind of lung disease it is, information as to the level (severity) of a lung disease, or progress information (degree of progression) of a disease.

The classifier 113 can classify a scalogram by unsupervised learning. The classifier 113 can classify which dynamic images are similar from a plurality of scalograms. In addition, the classifier 113 can perform classification based on a frequency spectrum by, for example, a principal component analysis (dimension compression).

Since the doctor can grasp, by the classification, which past dynamic image the current dynamic image is similar to, the doctor can make a diagnosis of the current subject by using information on a subject in a past dynamic image.

The classifier 113 can classify a scalogram by supervised learning. For example, the classifier 113 can perform classification based on machine learning using teacher data, such as a regression analysis or deep learning. For example, the teacher data is a dynamic image of a healthy patient, a dynamic image of a patient with mild pulmonary embolism, a dynamic image of a patient with severe emphysema, or the like. The classifier 113 may classify which scalogram of teacher data the scalogram of a dynamic image of a subject is similar to.

The classification makes it possible to provide information on a disease, for example, information on the possibility of a disease, information on the level (severity) of a certain disease, or the like for the current dynamic imaging.

Scalograms of at least two different regions of interest (ROIs) are compared to determine the relevance (coherence). For example, a scalogram of the pulmonary artery is compared with a scalogram of the lung field, and when the same frequency component(s) is/are present at the same time (or a time delayed by a predetermined time), it is determined (classified) that the coherence is high.

When the coherence is high, it can be estimated that the blood in the pulmonary artery smoothly flows to the lung field, whereas when the coherence is low, it can be estimated that the blood in the pulmonary artery does not smoothly flow to the lung field due to some reasons. It can be estimated that the reason why the blood does not flow smoothly is that a lung disease exists. That is, information useful for a lung disease can be provided by comparing scalograms of different regions and based on classification based on the relevance (coherence).

Based on a frequency spectrum generated from a scalogram, it is possible to grasp a feature obtained by performing the non-stationary spectrum analysis for dynamic imaging. A feature obtained by performing the non-stationary spectrum analysis may be grasped based on the intensity of an arbitrary frequency region of a frequency spectrum. In a case where the intensity of a specific frequency is high, it can be estimated that resonance and/or reflection is/are occurring in blood. The reason why resonance and/or reflection occur(s) in blood is that the tissue of the lungs may not be uniform. That is, in the classification based on a frequency spectrum, for example, in a case where the intensity of a high frequency is increased in a frequency spectrum, for example, useful information on a lung disease can be provided.

A frequency spectrum can also be learned and classified. In the same manner as the learning of a scalogram, a frequency spectrum can be classified based on unsupervised learning or supervised learning.

For example, in a case where the signal intensity of a specific frequency is high in a frequency spectrum, it is possible to provide information that there is a possibility of emphysema or interstitial pneumonia. In addition, in a case where a frequency close to the frequency of a heartbeat is high only in a specific region in a frequency spectrum, it is possible to provide information that there is a possibility of pulmonary embolism.

Based on dynamic images of the same subject at different dates and times, information on the course of a disease can be provided.

Depending on a disease to be diagnosed, data to be classified is optimized. It can be said that the optimization corresponds to preprocessing of machine learning. For example, necessary data is selected according to the disease. For example, a different scalogram of a specific ROI is selected in a case where a disease A is to be diagnosed, and a frequency spectrum is selected in a case where a disease B is to be diagnosed. The processing circuit 110 may calculate only characteristic data to be selected, or may calculate every characteristic data and select one from the every characteristic data.

The classifier 113 performs, based on optimized data, classification based on the coherence of a scalogram of a specific ROI and performs classification based on a frequency spectrum.

Since the heart is imaged together with the lungs in a dynamic image obtained by imaging the chest, not only a blood vessel of the lungs but also a blood vessel of the heart can be analyzed. For example, useful information on a heart disease can be provided by performing the non-stationary spectrum analysis on the dilation and contraction of the atria and the ventricles and the dilation and contraction of the aorta.

In addition, when dynamic imaging is performed on the brain, a blood vessel in the brain can be analyzed, and thus, useful information on a brain disease can be provided.

In the case of dynamic imaging of the lungs (heart) of a subject, a disease to be diagnosed is a circulatory disease, for example, a lung disease, a heart disease, or a brain disease. Examples of the lung disease include the presence or absence and the degree (level) of emphysema, subtypes of interstitial pneumonia, pulmonary embolism, peripheral pulmonary embolism, and pleural effusion. Examples of the heart disease include heart failure or atrial fibrillation.

For example, it is possible to provide useful information for: discrimination between heart failure and pneumonia; a diagnosis as to whether heart failure is heart failure with reduced ejection fraction (HFrEF) or heart failure with preserved ejection fraction (HFpEF); screening for performing spirometry; discrimination between atelectasis and pleural effusion; a diagnosis of the degree of adhesion and thickness of pleurisy; and an estimation of the left atrial pressure.

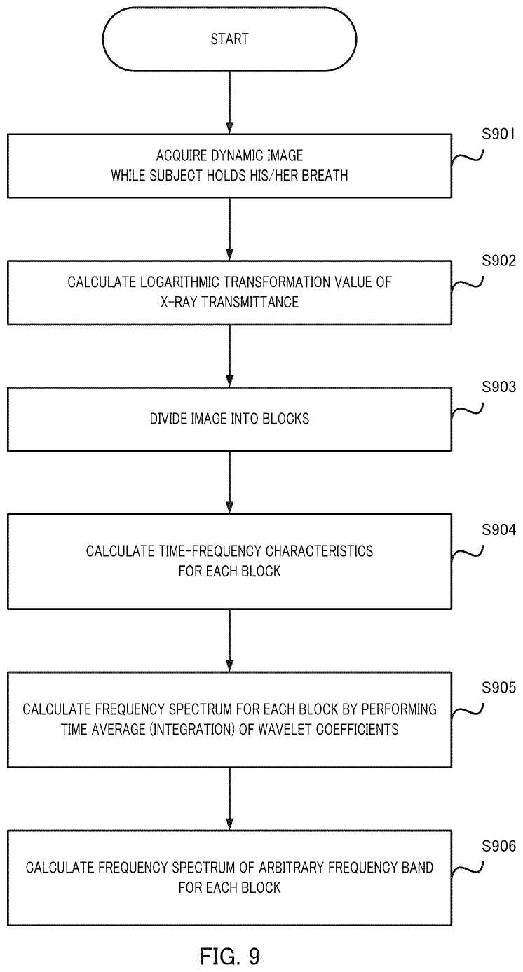

FIG. 9 illustrates a flowchart of a program that is executed by the processing circuit 110.

The acquirer 111 of the processing circuit 110 acquires a dynamic image that has been captured while a subject holds his/her breath (step S901). The dynamic image may be acquired from an external apparatus such as a dynamic imaging apparatus via the communicator 130 or may be acquired from the memory 140. The dynamic image is acquired as the intensity of each pixel constituting a detector of an X-ray imaging apparatus. In a case where the size of the pixel of the detector is, for example, 0.4 mm square, the acquired dynamic image has the intensity of each pixel with the size of 0.4 mm square.

The analyzer 112 of the processing circuit 110 calculates a logarithmic transformation value by logarithmically transforming the intensity of the acquired dynamic image (step S902). Since the intensity of X-rays is a value having the density ρ of the lungs as an index as expressed by the equation (3) described above, a linear value with respect to the density ρ of the lungs can be obtained by performing the logarithmic transformation.

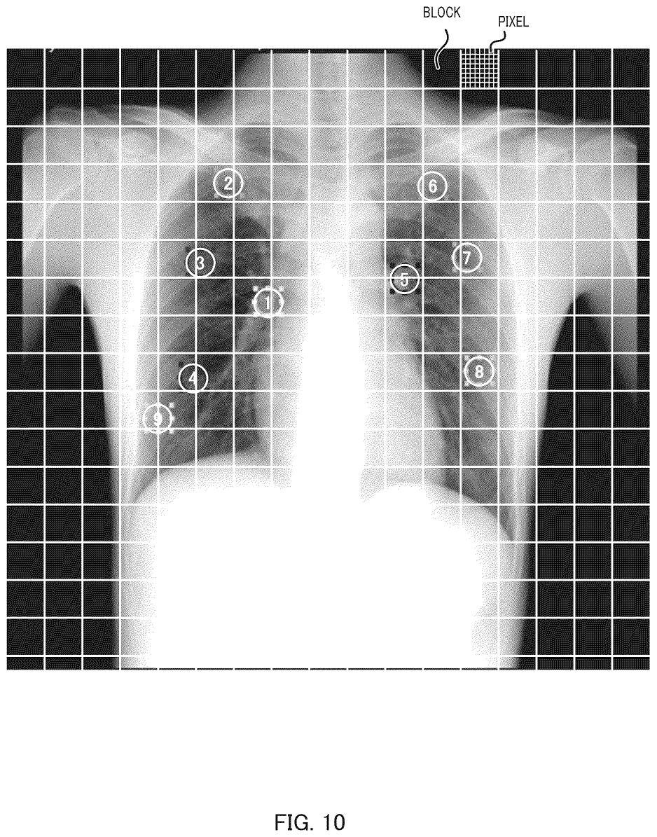

The analyzer 112 of the processing circuit 110 divides the dynamic image acquired in step S901 into blocks (step S903). The analyzer 112 converts the dynamic image into, for example, blocks having a block size of 3 mm square. For example, it is possible to convert the dynamic image into blocks having each block size of 3.2 mm by calculating the average for 8×8 pixels each time from data for each pixel (0.4 mm square) acquired in step S901. By dividing the dynamic image into blocks, the influence of noise can be reduced. In the division into blocks, the S/N (intensity resolution) increases when the block size is increased (the spatial resolution is decreased), whereas the S/N (intensity resolution) decreases when the block size is decreased (the spatial resolution is increased). That is, the size of the block size is determined according to at least one of the spatial resolution and the intensity resolution that are desired to be acquired. FIG. 10 is a diagram illustrating an image of division into blocks.

The analyzer 112 of the processing circuit 110 performs the non-stationary spectrum analysis for each of the blocks into which the dynamic image has been divided to calculate the time-frequency characteristics (scalogram) for each block (step S904). The non-stationary spectrum analysis is, for example, the wavelet transform, the windowed FFT, or the Wigner distribution.

The analyzer 112 of the processing circuit 110 calculates, for each block, the maximum value, the average value, or the sum of the wavelet coefficients (intensities) for the respective frequency components with respect to the time from the first time to the second time of the calculated scalogram, and generates (calculates) a frequency spectrum for each block (step S905). For example, in a case where a dynamic image includes 120 frames in total, the processing circuit 110 may calculate the maximum value, the average value, or the sum of the intensities based on the frames from the 21st frame to the 100th frame. The processing circuit 110 displays the generated frequency spectrum for each block with the horizontal axis representing frequency and the vertical axis representing intensity. For example, in response to selection of a ROI displayed in FIG. 3, the processing circuit 110 displays the frequency spectrum of the selected ROI. The processing circuit 110 displays, as the intensity, at least one of the maximum value, the average value, and the sum from the first time to the second time. The processing circuit 110 may display, as the intensity to be displayed, the intensity of each frequency or a ratio (frequency distribution of energy) of the intensity of each frequency to the intensities of all frequencies. The intensities of a plurality of ROIs may be displayed.

The analyzer 112 of the processing circuit 110 acquires an arbitrary spectrum (the intensity of an arbitrary frequency band) of a frequency spectrum for each calculated block (step S906). The analyzer 112 displays the acquired intensity for each block as an image of the chest. The frequency band to be acquired is selectable. A doctor can make a diagnosis of a lung disease based on the intensity of each block.

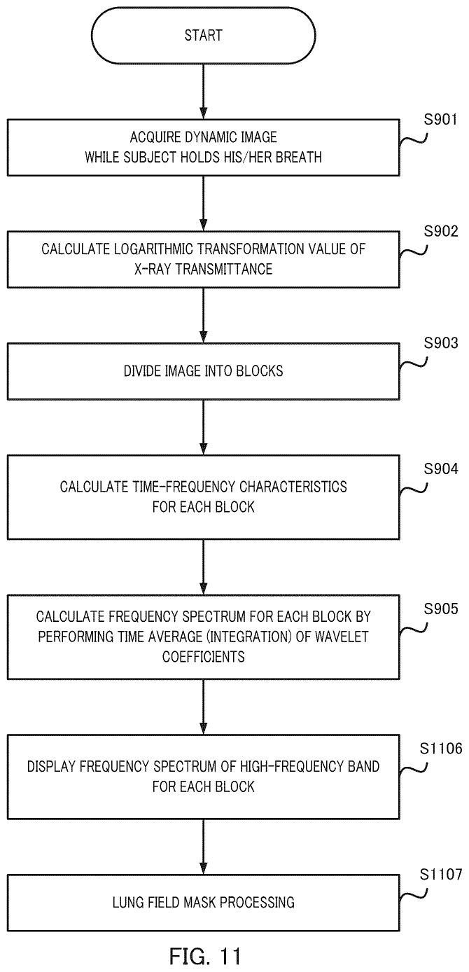

FIG. 11 illustrates an example of a flowchart of a program for diagnosing emphysema, which is executed by the processing circuit 110.

The processing from step S901 to step S905 is the same as that in FIG. 9, and thus, the description thereof will be omitted.

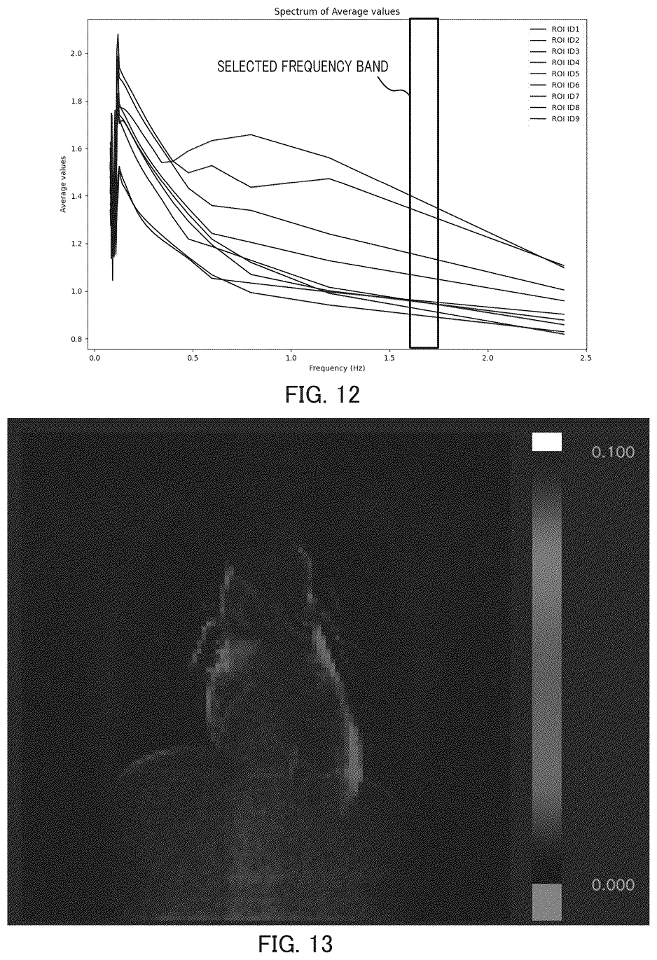

The analyzer 112 of the processing circuit 110 acquires, for the frequency spectrum of each block calculated in step S905, the intensity of a high-frequency band (for example, a frequency band higher than the cardiac motion) for each block (step S1106). The intensity of the frequency band is calculated as an average or a sum of the selected frequency bands. FIG. 12 is a diagram illustrating an example in step S1106 in which a high-frequency band is selected from the frequency spectrum for each block calculated in step S905. FIG. 13 is a diagram illustrating an example in which the calculated intensity of the high-frequency band is illustrated for each block.

A signal having a frequency component higher than that of the cardiac motion can be estimated to be a signal generated when a signal induced by the cardiac motion collides with air and is reflected. Accordingly, by acquiring the intensity of the high-frequency band for each block, it is possible to obtain information useful for diagnosing in which block much air exists, that is, which portion has emphysema.

The analyzer 112 of the processing circuit 110 performs lung field mask processing (step S1107). The lung field mask processing is processing for extracting only a region of the lung field. By deleting regions not related to the diagnosis, only information related to the determination of a lung disease can be provided to the doctor.

### MODIFICATIONS

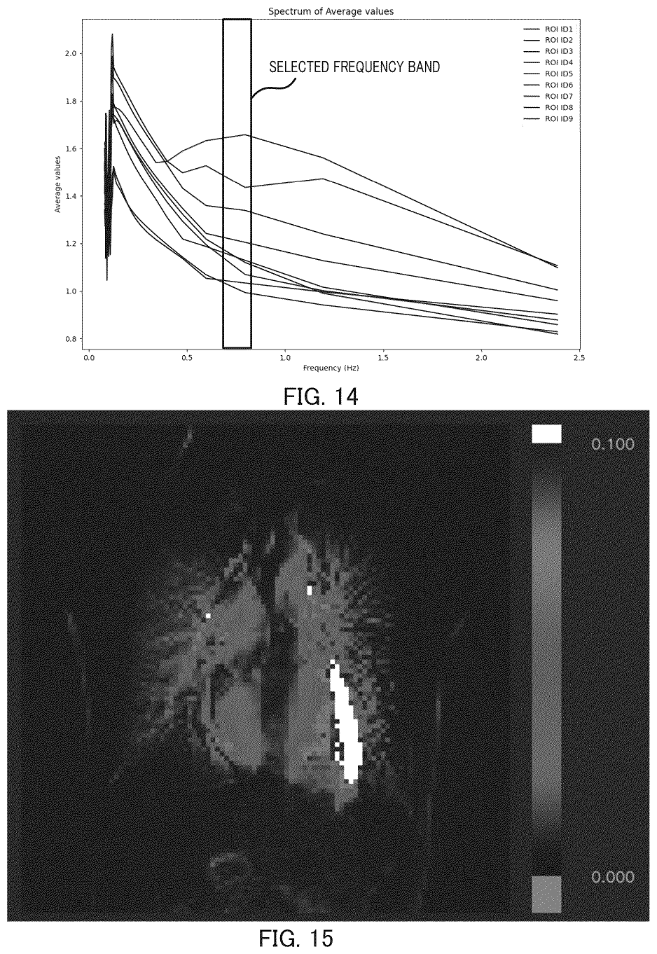

On the other hand, it is assumed that when the lungs become fibrotic, the lungs become hard and resonance occurs in a low-frequency band. Accordingly, the analyzer 112 of the processing circuit 110 acquires a frequency spectrum of a low-frequency band (for example, a frequency band lower than the cardiac motion) for each calculated block, and thus, it is possible to obtain information useful for diagnosing in which portion interstitial pneumonia (pulmonary fibrosis) is occurring. FIG. 14 is a diagram illustrating, as step S906, an example in which a low-frequency band is selected from the spectrum for each block calculated in step S905. FIG. 15 is a diagram illustrating an example in which the calculated intensity of the low-frequency band is illustrated for each block.

In step S906 of FIG. 9, the frequency band of the frequency spectrum calculated for each block can be selected according to the type of the disease.

Although an embodiment has been described above with reference to the accompanying drawings, the present disclosure is not limited to such an example. It is obvious that a person skilled in the art can conceive of various change examples or modification examples within the scope described in the claims. It is to be understood that such change examples or modification examples also belong to the technical scope of the present disclosure. Further, the constituent elements in the embodiment may be arbitrarily combined without departing from the spirit of the present disclosure.
(1) A dynamic image analysis apparatus according to an embodiment of the present disclosure includes: a hardware processor that performs an acquisition of a dynamic image of an imaged person who has been imaged by dynamic imaging; and a hardware processor that performs an analysis, which is a non-stationary spectrum analysis, on the dynamic image having been acquired by the hardware processor that performs the acquisition.
(2) In the dynamic image analysis apparatus according to an embodiment of the present disclosure, in the dynamic image analysis apparatus of (1), the non-stationary spectrum analysis is an analysis based on a non-stationary signal of a biological signal acquired from the dynamic image.
(3) In the dynamic image analysis apparatus according to an embodiment of the present disclosure, in the dynamic image analyzing apparatus of (1), the dynamic image captures an influence of a signal associated with cardiac motion.
(4) In the dynamic image analysis apparatus according to an embodiment of the present disclosure, in the dynamic image analysis apparatus of (2), the non-stationary signal is a signal in which at least one of a frequency and/or an amplitude changes.
(5) In the dynamic image analysis apparatus according to an embodiment of the present disclosure, in the dynamic image analysis apparatus of (1), the non-stationary spectrum analysis is a time-frequency analysis, a multi-resolution analysis, or an analysis technique in which the time-frequency analysis and the multi-resolution analysis are combined, where the time-frequency analysis, the multi-resolution analysis, and the analysis technique is to be performed on a non-stationary signal.
(6) In the dynamic image analysis apparatus according to an embodiment of the present disclosure, in the dynamic image analysis apparatus of (4) or (5), the non-stationary spectrum analysis is a wavelet transform, a windowed fast Fourier transform, or a transform using a Wigner distribution.
(7) In the dynamic image analysis apparatus according to an embodiment of the present disclosure, in the dynamic image analysis apparatus of (6), the non-stationary spectrum analysis is the wavelet transform, and the hardware processor that performs the analysis outputs a scalogram subjected to the wavelet transform.
(8) In the dynamic image analysis apparatus according to an embodiment of the present disclosure, in the dynamic image analysis apparatus of (6), the non-stationary spectrum analysis is the wavelet transform, and the hardware processor that performs the analysis outputs a frequency spectrum generated from a scalogram subjected to the wavelet transform.
(9) In the dynamic image analysis apparatus according to an embodiment of the present disclosure, in the dynamic image analysis apparatus of (1), the dynamic image is a dynamic image of the imaged person captured by dynamic imaging while the imaged person holds breath of the image person.
(10) In the dynamic image analysis apparatus according to an embodiment of the present disclosure, in the dynamic image analysis apparatus of (1), the hardware processor that performs the analysis divides the dynamic image, which has been acquired by the hardware processor that performs the acquisition, into blocks, and the non-stationary spectrum analysis is a non-stationary spectrum analysis on the dynamic image for each of the blocks into which the dynamic image has been divided.
(11) In the dynamic image analysis apparatus according to an embodiment of the present disclosure, in the dynamic image analysis apparatus of (10), a block size of the blocks is determined according to at least one of a spatial resolution and/or an intensity resolution, where the spatial resolution and the intensity resolution are desired to be acquired.
(12) In the dynamic image analyzing apparatus according to an embodiment of the present disclosure, in the dynamic image analysis apparatus of (8), the hardware processor that performs the analysis calculates an intensity of a predetermined band of the frequency spectrum.
(13) In the dynamic image analysis apparatus according to an embodiment of the present disclosure, in the dynamic image analysis apparatus of (12), the predetermined band is selectable.
(14) A dynamic image analysis method according to an embodiment of the present disclosure includes: acquiring a dynamic image of an imaged person who has been imaged by dynamic imaging; and performing a non-stationary spectrum analysis on the acquired dynamic image.
(15) In the dynamic image analysis method according to an embodiment of the present disclosure, in the dynamic image analysis method of (14), the non-stationary spectrum analysis is an analysis based on a non-stationary signal of a biological signal acquired from the dynamic image.
(16) In the dynamic image analysis method according to an embodiment of the present disclosure, in the dynamic image analysis method of (14), the dynamic image captures an influence of a signal associated with cardiac motion.
(17) In the dynamic image analysis method according to an embodiment of the present disclosure, in the dynamic image analysis method of (15), the non-stationary signal is a signal in which at least one of a frequency and/or an amplitude changes.
(18) In the dynamic image analysis method according to an embodiment of the present disclosure, in the dynamic image analysis method of (14), the non-stationary spectrum analysis is a time-frequency analysis, a multi-resolution analysis, or an analysis technique in which the time-frequency analysis and the multi-resolution analysis are combined, where the time-frequency analysis, the multi-resolution analysis, and the analysis technique is to be performed on a non-stationary signal.
(19) In the dynamic image analysis method according to an embodiment of the present disclosure, in the dynamic image analysis method of (17) or (18), the non-stationary spectrum analysis is a wavelet transform, a windowed fast Fourier transform, or a transform using a Wigner distribution.
(20) In the dynamic image analysis method according to an embodiment of the present disclosure, in the dynamic image analysis method of (19), the non-stationary spectrum analysis is the wavelet transform, and a scalogram subjected to the wavelet transform is outputted.
(21) In the dynamic image analysis method according to an embodiment of the present disclosure, in the dynamic image analysis method of (19), the non-stationary spectrum analysis is the wavelet transform, and a frequency spectrum generated from a scalogram subjected to the wavelet transform is outputted.
(22) In the dynamic image analysis method according to an embodiment of the present disclosure, in the dynamic image analysis method of (14), the dynamic image is a dynamic image of the imaged person captured by dynamic imaging while the imaged person holds breath of the image person.
(23) In the dynamic image analysis method according to an embodiment of the present disclosure, in the dynamic image analysis method of (14), the non-stationary spectrum analysis is a non-stationary spectrum analysis on the dynamic image, which has been acquired and has been divided into blocks, for each of the blocks.
(24) In the dynamic image analysis method according to an embodiment of the present disclosure, in the dynamic image analysis method of (23), a block size of the blocks is determined according to at least one of a spatial resolution and/or an intensity resolution, where the spatial resolution and the intensity resolution are desired to be acquired.
(25) In the dynamic image analysis method according to an embodiment of the present disclosure, in the dynamic image analysis method of (21), an intensity of a predetermined band of the frequency spectrum is calculated.
(26) In the dynamic image analysis method according to an embodiment of the present disclosure, in the dynamic image analysis method of (25), the predetermined band is selectable.
(27) In a non-transitory computer-readable recording medium storing a dynamic image analysis program according to an embodiment of the present disclosure, the dynamic image analysis program causes a computer to execute: acquiring a dynamic image of an imaged person who has been imaged by dynamic imaging; and performing a non-stationary spectrum analysis on the acquired dynamic image.
(28) In the non-transitory computer-readable recording medium storing the dynamic image analysis program according to an embodiment of the present disclosure, in the non-transitory computer-readable recording medium storing the program of (27), the non-stationary spectrum analysis is an analysis based on a non-stationary signal of a biological signal acquired from the dynamic image.
(29) In the non-transitory computer-readable recording medium storing the dynamic image analysis program according to an embodiment of the present disclosure, in the non-transitory computer-readable recording medium storing the program of (27), the dynamic image captures an influence of a signal associated with cardiac motion.
(30) In the non-transitory computer-readable recording medium storing the dynamic image analysis program according to an embodiment of the present disclosure, in the non-transitory computer-readable recording medium storing the program of (28), the non-stationary signal is a signal in which at least one of a frequency and/or an amplitude changes.
(31) In the non-transitory computer-readable recording medium storing the dynamic image analysis program according to an embodiment of the present disclosure, in the non-transitory computer-readable recording medium storing the program of (27), the non-stationary spectrum analysis is a time-frequency analysis, a multi-resolution analysis, or an analysis technique in which the time-frequency analysis and the multi-resolution analysis are combined, where the time-frequency analysis, the multi-resolution analysis, and the analysis technique is to be performed on a non-stationary signal.
(32) In the non-transitory computer-readable recording medium storing the dynamic image analysis program according to an embodiment of the present disclosure, in the non-transitory computer-readable recording medium storing the program of (30) or (31), the non-stationary spectrum analysis is a wavelet transform, a windowed fast Fourier transform, or a transform using a Wigner distribution.
(33) In the non-transitory computer-readable recording medium storing the dynamic image analysis program according to an embodiment of the present disclosure, in the non-transitory computer-readable recording medium storing the program of (32), the non-stationary spectrum analysis is the wavelet transform, and a scalogram subjected to the wavelet transform is outputted.
(34) In the non-transitory computer-readable recording medium storing the dynamic image analysis program according to an embodiment of the present disclosure, in the non-transitory computer-readable recording medium storing the program of (32), the non-stationary spectrum analysis is the wavelet transform, and a frequency spectrum generated from a scalogram subjected to the wavelet transform is outputted.
(35) In the non-transitory computer-readable recording medium storing the dynamic image analysis program according to an embodiment of the present disclosure, in the non-transitory computer-readable recording medium storing the program of (27), the dynamic image is a dynamic image of the imaged person captured by dynamic imaging while the imaged person holds breath of the image person.
(36) In the non-transitory computer-readable recording medium storing the dynamic image analysis program according to an embodiment of the present disclosure, in the non-transitory computer-readable recording medium storing the program of (27), the non-stationary spectrum analysis is a non-stationary spectrum analysis on the dynamic image, which has been acquired and has been divided into blocks, for each of the blocks.
(37) In the non-transitory computer-readable recording medium storing a dynamic image analysis program according to an embodiment of the present disclosure, in the non-transitory computer-readable recording medium storing the program of (36), a block size of the blocks is determined according to at least one of a spatial resolution and/or an intensity resolution, where the spatial resolution and the intensity resolution are desired to be acquired.
(38) In the non-transitory computer-readable recording medium storing the dynamic image analysis program according to an embodiment of the present disclosure, in the non-transitory computer-readable recording medium storing the program of (34), an intensity of a predetermined band of the frequency spectrum is calculated.
(39) In the non-transitory computer-readable recording medium storing the dynamic image analysis program according to an embodiment of the present disclosure, in the non-transitory computer-readable recording medium storing the program of (38), the predetermined band is selectable.

### INDUSTRIAL APPLICABILITY

The present disclosure is useful for a dynamic image analysis apparatus, a method, and a non-transitory computer-readable recording medium storing a program.

Although embodiments of the present invention have been described and illustrated in detail, the disclosed embodiments are made for purpose of illustration and example only and not limitation. The scope of the present invention should be interpreted by terms of the appended claims.

What is claimed is:

1. A dynamic image analysis apparatus, comprising:
a hardware processor that performs an acquisition of a dynamic image of an imaged person who has been imaged by dynamic imaging; and
a hardware processor that performs an analysis on the dynamic image having been acquired by the hardware processor that performs the acquisition, the analysis being a non-stationary spectrum analysis.

2. The dynamic image analysis apparatus according to claim 1, wherein
the non-stationary spectrum analysis is an analysis based on a non-stationary signal of a biological signal acquired from the dynamic image.

3. The dynamic image analysis apparatus according to claim 1, wherein
the non-stationary spectrum analysis is a time-frequency analysis, a multi-resolution analysis, or an analysis technique, the analysis technique being an analysis technique in which the time-frequency analysis and the multi-resolution analysis are combined, the time-frequency analysis, the multi-resolution analysis, and the analysis technique being to be performed on a non-stationary signal.

4. The dynamic image analysis apparatus according to claim 3, wherein
the non-stationary spectrum analysis is a wavelet transform, a windowed fast Fourier transform, or a transform using a Wigner distribution.

5. The dynamic image analysis apparatus according to claim 4, wherein:
the non-stationary spectrum analysis is the wavelet transform, and
the hardware processor that performs the analysis outputs a scalogram or a frequency spectrum, the scalogram being subjected to the wavelet transform, the frequency spectrum being generated from the scalogram.

6. The dynamic image analysis apparatus according to claim 1, wherein:
the hardware processor that performs the analysis divides the dynamic image into blocks, the dynamic image having been acquired by the hardware processor that performs the acquisition, and
the non-stationary spectrum analysis is a non-stationary spectrum analysis on the dynamic image for each of the blocks into which the dynamic image has been divided.

7. The dynamic image analysis apparatus according to claim 6, wherein
a block size of the blocks is determined according to at least one of a spatial resolution and/or an intensity resolution, the spatial resolution and the intensity resolution being desired to be acquired.

8. The dynamic image analysis apparatus according to claim 5, wherein
the hardware processor that performs the analysis calculates an intensity of a predetermined band of the frequency spectrum.

9. The dynamic image analysis apparatus according to claim 8, wherein
the predetermined band is selectable.

10. A dynamic image analysis method, comprising:
acquiring a dynamic image of an imaged person who has been imaged by dynamic imaging; and
performing a non-stationary spectrum analysis on the dynamic image having been acquired.

11. The dynamic image analysis method according to claim 10, wherein
the non-stationary spectrum analysis is an analysis based on a non-stationary signal of a biological signal acquired from the dynamic image.

12. The dynamic image analysis method according to claim 10, wherein
the non-stationary spectrum analysis is a time-frequency analysis, a multi-resolution analysis, or an analysis technique, the analysis technique being an analysis technique in which the time-frequency analysis and the multi-resolution analysis are combined, the time-frequency analysis, the multi-resolution analysis, and the analysis technique being to be performed on a non-stationary signal.

13. The dynamic image analysis method according to claim 12, wherein
the non-stationary spectrum analysis is a wavelet transform, a windowed fast Fourier transform, or a transform using a Wigner distribution.

14. The dynamic image analysis method according to claim 13, wherein:
the non-stationary spectrum analysis is the wavelet transform, and
a scalogram or a frequency spectrum is outputted, the scalogram being subjected to the wavelet transform, the frequency spectrum being generated from the scalogram.

15. The dynamic image analysis method according to claim 10, wherein
the non-stationary spectrum analysis is a non-stationary spectrum analysis on the dynamic image for each of blocks of the dynamic image, the dynamic image having been acquired and having been divided into the blocks.

16. The dynamic image analysis method according to claim 15, wherein
a block size of the blocks is determined according to at least one of a spatial resolution and/or an intensity resolution, the spatial resolution and the intensity resolution being desired to be acquired.

17. The dynamic image analysis method according to claim 14, wherein
an intensity of a predetermined band of the frequency spectrum is calculated.

18. The dynamic image analysis method according to claim 17, wherein
the predetermined band is selectable.

19. A non-transitory computer-readable recording medium storing a dynamic image analysis program that causes a computer to execute:
acquiring a dynamic image of an imaged person subject who has been imaged by dynamic imaging; and
performing a non-stationary spectrum analysis on the dynamic image having been acquired.
